(19) United States
(12) Patent Application Publication
(10) Pub. No.: US 2002/0035747 A1
(43) Pub. Date: Mar. 28, 2002

(54) Briefs for supporting an absorbent article

(76) Inventors: Liljana Kusibojoska, Hisings Karra (SE); Peter Ronnberg, Molndal (SE)

Correspondence Address: Ronald L. Grudziecki, BURNS, DOANE, SWECKER & MATHIS, L.L.P., P.O. Box 1404, Alexandria, VA, 22313-1404, US

(21) Appl. No.: 09/955,019

(22) Filed: Sep. 19, 2001

Related U.S. Application Data
(60) Provisional application No. 60/234,972, filed on Sep. 25, 2000.

(51) Int. Cl. A41B 9/12; A61F 13/15

(52) U.S. Cl. 2/400; 604/385.03; 2/919

(57) ABSTRACT

Briefs (400; 500) for supporting an absorbent article have a front portion, a rear portion and an intermediate crotch portion, two leg openings, and a waist opening. The briefs also have a fastening member (401; 501a,b) which is arranged to cooperate with a corresponding fastening member (325; 326) on an absorbent article for the purpose of securing the article in the briefs (400; 500). The fastening member arranged in the briefs has a surface provided with fastening loops (405; 505), the fastening loops (405; 505) being designed to cooperate with a fastening member comprising hook members, and the fastening loops (405; 505) being arranged in a number of chain-like rows of loops (505), each formed by a continuous thread which penetrates through a layered carrier material (402; 502).

### CROSS-REFERENCE TO RELATED APPLICATIONS

The present application claims the benefit of U.S. Provisional Application No. 60/234,972, filed in the United States on Sep. 25, 2000. The contents of U.S. Provisional Application No. 60/234,972 are hereby incorporated herein by reference.

### BACKGROUND OF INVENTION

1. Technical Field

The present invention relates to briefs for supporting an absorbent article, and in particular to briefs designed with a front portion, a rear portion and an intermediate crotch portion and having two leg openings and a waist opening. The briefs include a fastening member which is arranged to cooperate with a corresponding fastening member on an absorbent article for the purpose of securing the article in the briefs.

2. Description of Related Art

It has become increasingly common for adhesive fastening members to be replaced by mechanical fastening arrangements of the hook-and-loop type. Such a fastening arrangement comprises a fastening member having a surface provided with loop elements and designed to cooperate with a fastening member having a surface provided with hooks or hook-like projections which can hook securely on the surface provided with loop elements.

However, for the type of absorbent article which is secured inside a pair of briefs, it has not as yet been possible to offer an acceptable alternative. This is because an absorbent article, with a hook and loop fastening arrangement, requires special briefs which allow the hook members on the fastening arrangement to fasten to the briefs.

Fastening members of the loop type which have been available to date generally consist of a laminate of a carrier material, for example a plastic film or a nonwoven material and a loop material, for example a loosely knitted or woven textile material or a loosely bonded nonwoven material. However, a disadvantage of fastening members comprising a plastic layer is that they are not permeable to air and vapor, which means that such a fastening member is airtight and uncomfortable to wear against the skin. This is particularly so if large areas of skin are covered by the airtight material, as is the case when the material is used in a pair of briefs. For reasons of comfort, it is also desirable that the material of the briefs is soft and gentle against the user's skin, that it does not have sharp or hard edges which can chafe the skin, and also that the material has a certain breathability and is able to let body moisture and air pass through. It is also desirable for the loop material to be inexpensive so that it can be used for disposable briefs, i.e., briefs which are discarded after a single use and are not washed or reused.

### OBJECTS AND SUMMARY

An object of the invention is to make available disposable briefs which comprise a fastening member provided with loops, and which briefs can be made soft and comfortable, air-permeable and vapor-permeable, and can be produced in a cost-effective manner.

Briefs designed according to the present invention, and of the type discussed above, are principally distinguished by the fact that the fastening member arranged in the briefs has a surface provided with fastening loops, the fastening loops being designed to cooperate with a fastening member comprising hook members, and the fastening loops are arranged in a number of chain-like rows of loops, each formed by a continuous thread which penetrates through a layered carrier material.

The fastening member provided with loops, included in the briefs according to the present invention, can advantageously be made by one or more rows of loops sewn into a layered carrier material. This affords several advantages. One advantage is that the fastening loops are anchored in a mechanical manner in the carrier material, which means that it is not necessary to use thermoplastic materials or other binders. However, it can be expedient for some component in either the thread and/or the carrier material to be thermoplastic, since it is then possible to secure cut-off or clipped-off thread ends by melting the thread end to the carrier material. Such melting can be carried out in conjunction with the fastening member being cut to the right shape and size. Alternatively, it is of course possible to secure the cut-off thread ends with adhesive, for example.

A further advantage of the fastening member described above is that it has a high degree of breathability since the carrier material is penetrated by the thread which forms the fastening loops. This makes the fastening member according to the invention especially suitable for use in combination with articles such as briefs which cover a large area of the skin surface when in use.

Since the fastening member can be produced by means of rows of loops being sewn into a carrier material, it is also possible to increase the bendability of a stiff material so that the material is thereby easier to bend along bend lines which run parallel with the rows of loops. Correspondingly, the stiffness of the material increases transversely to the rows of loops. This feature can be used to increase the stability in particularly exposed areas of the briefs. For example, the rows of loops can advantageously be arranged principally at right angles to the waist edge of the briefs, as a result of which the briefs easily bend round the user's waist, while reducing the likelihood that the material nearest to the waist edge will not bend, roll up or crease. These phenomena often occur when the person using the briefs is overweight, and they cause a number of problems. For example, the risk of body fluid leaking out of an article worn in the briefs and extending to the waist edge increases if the waist edge is folded out onto the outside of the briefs. It is of course also uncomfortable for the user if the waist edge is folded or in some other way deformed.

Suitable materials for use as carrier material for a fastening member provided with loops are layered materials such as plastic film, nonwoven, or a laminate of two or more layers of plastic film, nonwoven, tissue or the like. The carrier material expediently may consist of a single flexible layer of nonwoven with a basis weight of about 20 g/m². Particularly suitable materials are spunbond materials, but other nonwoven materials such as carded material, through-air-bonded material or the like can also be used, and also different laminates.

In a pair of briefs according to the invention, the fastening loops in the fastening member provided with the loops are arranged on the inside of the briefs and in contact with the user's skin. The briefs are in this case made completely or partially of material provided with loops. This means that the thread, in order to be comfortable against the user's skin, should be smooth and not too coarse. Preferably, the thread is not absorbent, so that liquid is not transported in the thread. However, it is possible to use absorbent thread material such as viscose and cotton. The absorption properties of the thread are of less importance in some designs. In addition, the absorption properties are unimportant in areas of the briefs which are at little risk of being wetted by body fluid. However, the thread is preferably made of nylon, polyester, polyethylene, polypropylene or the like. Voluminous threads comprising many essentially parallel fibers are preferred, and the thread's combined coarseness should preferably be approximately 20-90 deniers.

The fastening loops preferably have a length of 2-10 millimeters. The length of a fastening loop refers to the distance between two points of penetration in a row of loops.

The eyelets of the fastening member are preferably arranged in essentially parallel rows with a spacing of 1-10 mm between the rows. It can also be advantageous to arrange rows of loops in groups of two or more rows with a greater spacing between the groups than between the rows included in the groups. By grouping the rows of loops, it is possible to obtain zones with different properties, for example different fastening capacity, different breathability, different liquid impermeability and different stiffness. It is also possible to use the rows of loops to obtain esthetically attractive patterns at the same time as the desired function. The rows of loops can thus be distributed across the entire surface of the briefs material. Alternatively, the fastening loops can be provided only on those parts of the briefs expected to be used for fastening an absorbent article in the briefs. These parts of the briefs are the crotch portion and, depending on the size and shape of the absorbent article, in some cases also the areas inside of the waist edge of the briefs.

The briefs can also be made of different types of material in different areas. In this way, it is possible to produce briefs with areas which differ in terms of properties such as elasticity, stretchability, compressibility, breathability, absorption capacity or the like. According to the present invention, the loops may be sewn directly onto the fabric of the briefs, or the loops may be sewn onto a carrier material, which is then attached to the briefs. In addition, the briefs may include hooks and the absorbent article may include the loops.

### BRIEF DESCRIPTION OF THE FIGURES

The invention will now be described in greater detail with reference to the illustrative embodiments shown in the attached drawings, in which:

FIG. 1 shows a fastening member provided with loops,

FIG. 2 shows in detail a row of loops on the fastening member in FIG. 1,

FIG. 3 shows a plan view of an incontinence pad, seen from the side which is directed away from the user during use,

FIG. 4 shows a pair of briefs according to the invention for supporting an absorbent article of the type shown in FIG. 3, and

FIG. 5 shows a further embodiment of a pair of briefs according to the invention.

### DETAILED DESCRIPTION OF THE PREFERRED EMBODIMENTS

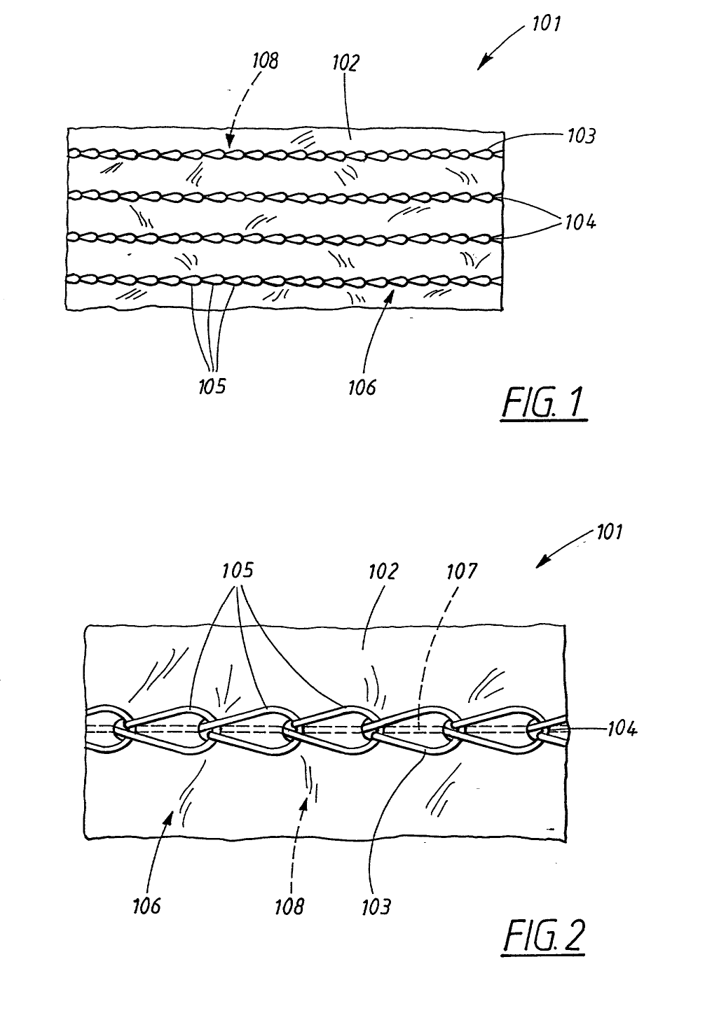

Referring to FIGS. 1 and 2, a fastening member 101 comprises a carrier material 102 provided with loops 105. The loops 105 are formed by a thread 103 forming a chain-like row 104 of fastening loops 105 on a first surface 106 of the carrier material 102. The fastening loops 105 are connected by a row of thread elements 107 which form straight stitches on the opposite, second surface 108 of the carrier material.

The carrier material 102 consists of a layer material, such as plastic film, nonwoven, or a laminate of two or more layers of plastic film, nonwoven, tissue or the like. Particularly preferred materials are, as has already been mentioned, soft and easily bendable nonwoven materials, for example of the spunbond type.

The thread 103 which forms the fastening loops 105 can be of any suitable material and can comprise synthetic and/or natural fibers. Thus, it is possible to use fibers of polyethylene, polypropylene, polyester, polyamide or two-component fibers such as cotton fibers, jute, flax, etc. The thread 103 is advantageously bulky and comprises in cross section a plurality of thin and substantially parallel fibers. It is also advantageous if the fibers have a certain curl since this increases the bulkiness and increases the thread's ability to cooperate with a hook member on an absorbent article.

The fastening member 101 comprises a plurality of rows 104 of fastening loops 105, as is shown in FIG. 1. Alternatively, the fastening member may include only one row of loops 105.

Although FIGS. 1 and 2 illustrate the loops 105 as being formed on a carrier material 102, the fastening member 101 may be created by forming the loops 105 directly onto the material of the briefs, without any intervening carrier material.

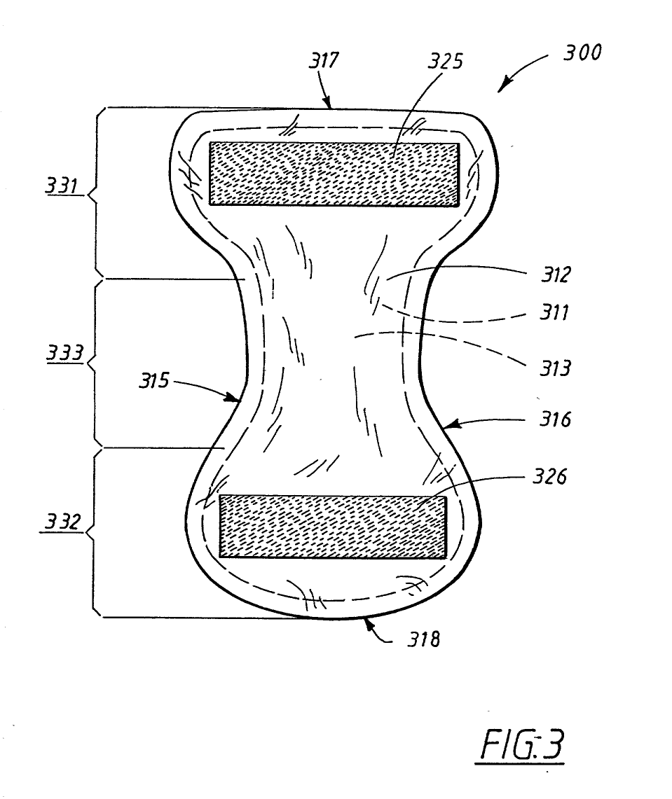

FIG. 3 shows an absorbent article in the form of an incontinence pad 300. The incontinence pad 300 comprises a liquid-permeable cover sheet 311, a liquid-impermeable cover sheet 312, and an absorption body 313 enclosed between the cover sheets 311, 312.

The incontinence pad 300 is substantially hourglass-shaped with a wide front portion 331, a wide rear portion 332, and a narrow crotch portion 333 located between the front portion 331 and the rear portion 332. The incontinence pad 300 also has two substantially longitudinal side edges 315, 316, a substantially transverse front edge 317, and a substantially transverse rear edge 318.

Two fastening members 325, 326 provided with hooks are arranged on the liquid-tight cover sheet 312.

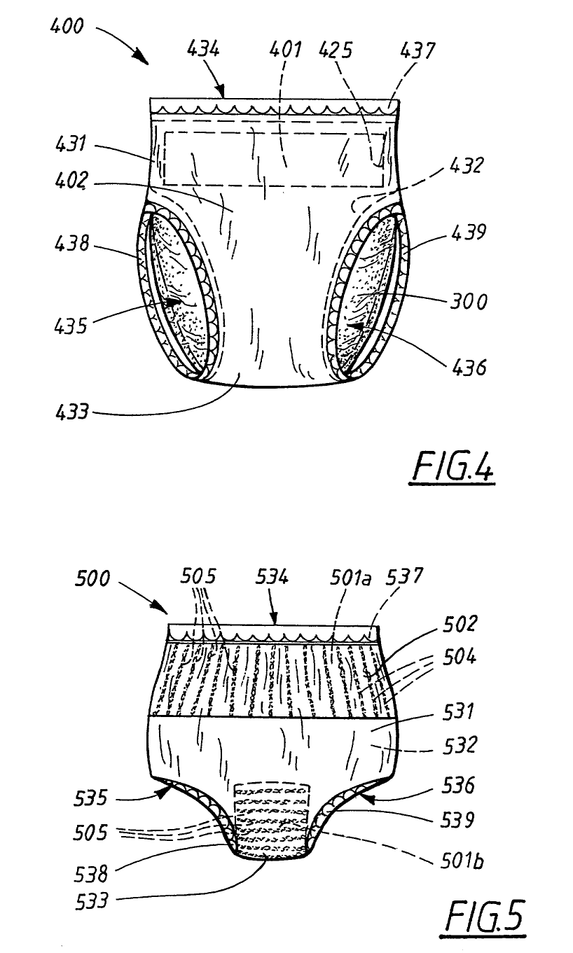

When using the incontinence pad 300 in FIG. 3, it is arranged inside a pair of briefs 400, as shown in FIG. 4. The briefs are of conventional shape and thus have a front portion 431, a rear portion 432 and an intermediate, narrower crotch portion 433. The briefs also have a waist opening 434 and two leg openings 435, 436. To fit securely in place during use, and to be adapted to the user's body, elastic members in the form of threads or bands are arranged as waist elastic 437 around the waist opening 434 and as leg elastic 438, 439 around the leg openings 435, 436.

The briefs 400 include a fastening member 401 provided with loops in the form of a number of rows of loops. The rows of loops can in this case be arranged in the longitudinal direction of the briefs, or in the transverse direction. The longitudinal direction is understood as the direction of a line extending between the front portion 431 and the rear portion 432 via the crotch portion 433. The transverse direction is at right angles to the longitudinal direction. It is of course possible to arrange the rows of loops in another direction in relation to the briefs, but this is generally less advantageous for manufacturing reasons. It is also possible to arrange the rows of eyelets as parallel straight rows, or as undulating rows, for example sinusoidal rows. The fastening member 401 provided with loops can be arranged across the whole inner surface of the briefs 400, as in FIG. 4, or can be applied only in those areas of the briefs which are expected to come into contact with corresponding fastening members provided with hooks on an absorbent article. The shape and location of the fastening member 401 can be chosen by means of sewing-in fastening loops only where they are desired. Thus, it is possible to control the liquid permeability, breathability, stiffness and fastening capacity of the material of the briefs by arranging fastening loops in different areas and with different stitch lengths and closeness between the rows of loops. It is also possible to sew the fastening loops in such a way that a decorative effect is obtained at the same time as the desired functional properties.

The fastening member 101 disclosed in FIGS. 1 and 2 is one example of a fastening member 401 that can be used in the briefs 400. And, as set forth above, the loops can be sewn directly into the material of the briefs, or may be sewn into a carrier material, which is then attached to the briefs.

FIG. 5 shows briefs 500 of principally the same type as those shown in FIG. 4. The briefs thus have a front portion 531, a rear portion 532 and a crotch portion 533 and have a waist opening 534 and two leg openings 535, 536 which are provided with waist elastic 537 and leg elastic 538, 539.

The briefs have fastening members 501a, 501b provided with loops only around the waist opening 534 on the front and rear portions 531, 532 and on the crotch portion 533. The fastening member 501a provided with loops at the waist opening 534 is arranged as a belt running around the entire circumference of the briefs, but can of course alternatively be in the form of one or more separate areas on the front portion 531 and/or the rear portion 532.

The fastening member 501a provided with loops at the waist opening 534 can be arranged on the inside of a conventional briefs material. However, it is advantageous if the part of the briefs 500 located nearest to the waist opening 534 consists of a perforated fastening member provided with loops of the type which has been described in connection with FIGS. 1 and 2, and if the carrier material 502 of the fastening member thus also constitutes the briefs material. The briefs material within the area of the fastening member 501a can in this case be the same material as in the rest of the briefs or consists of a separate material which has been joined to the rest of the briefs material, for example by welding, adhesive bonding or stitching.

The fastening member 501b in the crotch area 502 can also be in the form of a separately produced component which has been joined to the material of the briefs or can be part of the material of the briefs. An advantage of using the carrier material 502 of the fastening members 501a, 501b as briefs material is that a high degree of breathability can thereby be achieved since the carrier material 502 is perforated upon attachment of the fastening loops 505. However, at the crotch portion 533, it can be desirable for the briefs 500 to have a greater degree of impermeability in order to ensure that any liquid leaking out from an absorbent article cannot leak further from the briefs. For this reason, the second fastening member 501b can be arranged as a separate component on the inside of the briefs 500. It is of course possible to provide the crotch portion 533 of the briefs with a special liquid barrier layer, for example a plastic film, or a coating of liquid-repelling or liquid-tight material.

And, as set forth above, the loops can be sewn directly into the material of the briefs, or may be sewn into a carrier material, which is then attached to the briefs.

The invention has been described above in connection with a number of embodiments. However, the invention must not be seen as being limited to these, and instead further modifications are possible within the scope of the attached patent claims. For example, a fastening member provided with loops according to the invention can be used in conjunction with absorbent articles and briefs of other shapes and sizes than those which have been used in the illustrative embodiments. The shape, size and position of the fastening members can of course also be varied within the scope of the invention.

What is claimed is:

1. Briefs for supporting an absorbent article, said briefs having a front portion, a rear portion and an intermediate crotch portion and having two leg openings and a waist opening, and said briefs comprising a fastening member which is arranged to cooperate with a corresponding fastening member on an absorbent article for the purpose of securing the article in the briefs, the fastening member arranged in the briefs has a surface provided with fastening loops, said fastening loops being designed to cooperate with a fastening member comprising hook members, and said fastening loops being arranged in a plurality of rows of loops, each row formed by a continuous thread which penetrates through a carrier material.

2. The briefs as claimed in claim 1, wherein the carrier material comprises a nonwoven material with a basis weight of about 20 g/m².

3. The briefs as claimed in claim 1, wherein the briefs comprise the carrier material for the fastening loops.

4. The briefs as claimed in claim 1, wherein the fastening loops have a length of 1-10 mm.

5. The briefs as claimed in claim 1, wherein the rows of loops are uniformly distributed across the area provided with loops, and wherein the distance between the rows of loops is 1-10 mm.

6. The briefs as claimed in claim 1, wherein the fastening member provided with loops is arranged in the crotch portion of the briefs.

7. The briefs as claimed in claim 6, wherein the fastening member provided with loops is also arranged at the waist opening of the briefs.

8. The briefs as claimed in claim 1, wherein the thread which forms the rows of loops has a coarseness of 20-90 deniers.

9. The briefs as claimed in claim 1, wherein the fastening member provided with loops comprises at least one centrally arranged row of loops extending from the waist opening on the front portion of the briefs to the waist opening on the rear portion of the briefs.

10. The briefs as claimed in claim 1, wherein the carrier material is layered.

11. Briefs for supporting an absorbent article, said briefs having a front portion, a rear portion and an intermediate crotch portion and having two leg openings and a waist opening, and said briefs comprising a fastening member which is arranged to cooperate with a corresponding fastening member on an absorbent article for the purpose of securing the article in the briefs, the fastening member arranged in the briefs includes a plurality of fastening loops, said fastening loops being designed to cooperate with a fastening member comprising hook members, and said fastening loops being arranged in a plurality of rows of loops, each row formed by a continuous thread.

12. The briefs as claimed in claim 11, wherein the fastening loops have a length of 1-10 mm.

13. The briefs as claimed in claim 11, wherein the rows of loops are uniformly distributed across the area provided with loops, and wherein the distance between the rows of loops is 1-10 mm.

14. Briefs for supporting an absorbent article, said briefs having a front portion, a rear portion and an intermediate crotch portion and having two leg openings and a waist opening, and said briefs comprising a fastening member which is arranged to cooperate with a corresponding fastening member on an absorbent article for the purpose of securing the article in the briefs, the fastening member arranged in the briefs includes a layer of carrier material that is secured to the briefs and the layer of carrier material has a surface provided with a plurality of fastening loops, said fastening loops being designed to cooperate with a fastening member comprising hook members, and said fastening loops being arranged in a plurality of rows of loops, each row formed by a continuous thread which penetrates through the layer of carrier material.

15. The briefs as claimed in claim 14, wherein the layer of carrier material comprises a nonwoven material with a basis weight of about 20 g/m².

16. The briefs as claimed in claim 14, wherein the fastening loops have a length of 1-10 mm.

17. The briefs as claimed in claim 14, wherein the rows of loops are uniformly distributed across the area provided with loops, and wherein the distance between the rows of loops is 1-10 mm.

18. Briefs for supporting an absorbent article, said briefs having a front portion, a rear portion and an intermediate crotch portion and having two leg openings and a waist opening, and said briefs comprising a fastening member, the fastening member arranged in the briefs includes plurality of fastening loops, said fastening loops being designed to cooperate with a fastening member comprising hook members, and said fastening loops being arranged in a plurality of rows of loops, each row formed by a continuous thread.

19. The briefs as claimed in claim 18, further comprising a layer of carrier material for supporting the plurality of fastening loops, the layer of carrier material comprises a nonwoven material with a basis weight of about 20 g/m².

20. The briefs as claimed in claim 18, wherein the fastening loops have a length of 1-10 mm.
